(19) United States
(12) Patent Application Publication
(10) Pub. No.: US 2007/0135874 A1
(43) Pub. Date: Jun. 14, 2007

(54) Endoscope for therapeutic light delivery

(76) Inventor: John L. Bala, Pomfret Center, CT (US)

Correspondence Address: ALIX YALE & RISTAS LLP, 750 MAIN STREET, SUITE 1400, HARTFORD, CT, 06103, US

(21) Appl. No.: 11/603,760

(22) Filed: Nov. 22, 2006

Related U.S. Application Data
(60) Provisional application No. 60/738,773, filed on Nov. 22, 2005.

(51) Int. Cl. A61N 5/06

(52) U.S. Cl. 607/94

(57) ABSTRACT

An endoscope is configured to deliver therapeutically effective quantities and wavelengths of light to internal body tissues and cavities. One application for such an endoscope is the delivery of ultraviolet light (UV) to kill bacteria in body cavities or passages. An endoscope for therapeutic light delivery includes a light source for producing the desired light wavelengths, illumination optics transmissive of the therapeutic wavelength and configured to distribute the light in a therapeutically effective pattern, and a control mechanism that permits measured application of the therapeutic light. A light source compatible with the present invention is a xenon flash lamp. A xenon flash lamp emits short duration, high intensity, broad-spectrum bursts or pulses of light rich in UV and IR wavelengths.

### CROSS REFERENCE TO RELATED APPLICATIONS

This application claims the benefit of U.S. Provisional Application No. 60/738,773, filed Nov. 22, 2005.

### BACKGROUND OF THE INVENTION

1. Field of the Invention

The invention relates generally to endoscopes, which are widely used in the field of medicine, and in particular to an endoscope configured for therapeutic delivery of light to tissues and surfaces inside the body.

2. Description of the Related Art

Endoscopes are well-known medical instruments used to visualize the interior of a body cavity or organ. Endoscopes are used in a variety of operative procedures, including laparoscopic surgery where endoscopes are used to visually examine the peritoneal cavity.

Typical endoscopes are configured in the form of a probe having a distal end for insertion through a small incision in the body. The probe includes components for delivery of illumination light and collection of an image from inside the body. Optical fibers or optically transmissive material in a tubular formation typically provides illumination light delivery to a distal end of the probe. Imaging is typically carried out by an objective lens and relay optics that receive and deliver an image to the proximal end of the probe, which may be equipped with an eye piece or an electronic image capture device such as a CCD (charge coupled device) sensor array. Endoscope probes may be rigid or flexible, with the light delivery and image retrieval components configured accordingly. Flexible bundles of optical fibers are used to produce a flexible probe, while rigid probes may have fused optical fiber assemblies, rigid light pipes and/or imaging rods and lenses. The intended use of the endoscope dictates the length of the probe, the need for flexibility and the necessary image quality.

Various wavelengths of light have therapeutic purposes. Ultra violet (UV) light is known to destroy and disable pathogens on tissue and in blood. UV is also useful for fluorescence imaging. Infra red light can be used for cauterizing or to facilitate clotting. Other wavelengths are used to activate light sensitive medications.

The incidence of infection by drug resistant pathogens has increased dramatically in recent years. The most common environments for drug resistant infections are hospitals and other health care facilities. These infections are referred to as “nosocomial infections.” Those in health care facilities are typically susceptible to infection because they are weakened in some way and are being subjected to invasive medical procedures. The National Nosocomial Infections Surveillance System reported that 57% of health care-associated antibiotic resistant pathogens identified in clinically isolated infections were methicillin-resistant. Thirty to 50% of healthy adults are colonized with drug resistant pathogens, of which 10 to 20% are persistently colonized. Rates of staphylococcal colonization are high among patients with type 1 diabetes, intravenous drug users, hemodialysis patients and surgical patients.

Because the nose is the main ecological niche in human beings of nosocomial infections, an effective anti-microbial treatment for the nasal passages presents an opportunity to dramatically reduce such infections. Eradication of microbes from the nose and throat may prevent infection from spreading into the lungs and blood. Development of a device that could eradicate antibiotic resistant pathogens from the anterior nares could have a tremendous effect on reducing infections. This statement is supported by a confluence of articles regarding methicillin-resistant pathogens.

There is a need for practical and effective devices that will reduce or eliminate viable drug resistant pathogens in passages, cavities and tissues of humans. There is also need for devices configured to deliver therapeutic light into cavities, openings or tissues of the human body.

### SUMMARY OF THE INVENTION

Briefly stated, an endoscope according to aspects of the present invention is configured to deliver light to internal body cavities or tissues for therapeutic purposes. Different wavelengths of light, including ultraviolet and infrared are known to have therapeutic effects. For example, ultraviolet light is effective at killing or disabling many forms of bacteria and other infectious microbes, while infrared light can be used to cauterize body tissues. Other wavelengths of light can be employed to activate photosensitive medications or chemicals for therapeutic purposes.

An endoscope according to aspects of the present invention is configured to deliver therapeutically effective quantities and wavelengths of light to internal body tissues and cavities. One application for such an endoscope is the delivery of ultraviolet light (UV) to kill bacteria in body cavities or passages. For example heliobacter (H) pylori infection of the digestive tract is strongly associated with the development of ulcers. H pylori has recently been identified as a category I human carcinogen, playing a causative role in the development of gastric cancer. Endoscopic delivery of UV to the gastrointestinal tract may be employed to kill H pylori on and in tissues lining the digestive tract.

The basic components of an endoscope for therapeutic light delivery include: a light source for producing the desired light wavelengths; illumination optics transmissive of the therapeutic wavelength and configured to distribute the light in a therapeutically effective pattern; and a control mechanism to interrupt delivery of all or part of the therapeutic light.

An exemplary light source compatible with the present invention is a xenon flash lamp. A xenon flash lamp emits short duration, high intensity, broad-spectrum bursts or pulses of light. FIG. 1 illustrates the typical spectral distribution of a xenon flash lamp. Approximately 49% of the light energy from the xenon flash lamp is UV below 350 nanometers. FIG. 2 illustrates the time/power relationship in a xenon flash lamp. The light pulses quickly achieve a high intensity of approximately 100,000 watts and have a short duration of about 10 μS.

A further aspect of the present invention relates to selecting light transmission components for delivery of the therapeutic wavelength to the area of the body to be treated.

An exemplary endoscope probe includes light delivery components configured to enhance the quantity and intensity of light delivered to the distal end of the endoscope. A further aspect of the invention relates to a distribution optic at the distal end of the probe configured to distribute light in a therapeutically effective pattern to an area surrounding the distal end of the endoscope. The distribution optic may include prisms or a ring lens with internal reflecting surfaces can be employed for this purpose. The distribution optic may be a Fresnel-type lens.

An aspect of the present invention relates to a mechanism for interrupting or otherwise controlling application of the selected therapeutic light frequency. This can be accomplished by means of a filter, shutting off the light source or controlling the power and/or frequency (number of pulses per unit of time) of pulses of therapeutic light.

An alternative approach employs one light source for illumination purposes and a second light source to generate the therapeutic wavelengths. The illumination light source is energized according to a timing pattern to generate light used by an imaging system. The second light source is energized during a non-illumination, or dark time, in a controlled way to produce therapeutic light for delivery through the endoscope.

### BRIEF DESCRIPTION OF THE DRAWINGS

FIG. 1 is a graphical presentation of the spectral distribution of a Xenon flash lamp compared to the photopic response of a CCD camera and the spectral response of silicon;

FIG. 2 illustrates the time/power relationship for an exemplary Xenon flash lamp;

FIG. 3 is an exterior view of an endoscope for therapeutic light delivery according to aspects of the present invention;

FIG. 4 is an enlarged sectional view through the probe of an exemplary endoscope for therapeutic light delivery according to aspects of the present invention;

FIG. 5 is an enlarged exterior view of the body of an exemplary endoscope according to aspects of the present invention;

FIG. 6 is a cut-away view of the endoscope body of FIG. 5, showing the internal components and block diagram of further components;

FIG. 7 is a sectional view through the distal end of an endoscope probe according to aspects of the present invention;

FIG. 8 illustrates the illumination pattern and image field of an endoscope according to aspects of the present invention;

FIG. 9 is an enlarged end view of an endoscope probe, illustrating one possible configuration for a light distribution optic according to aspects of the present invention;

FIG. 10 is a side sectional view through the endoscope probe of FIG. 9;

FIG. 11 is an enlarged cross-sectional elevational view of the end of the optical viewing device employing the ring lens assembly of FIG. 1;

FIG. 12 is an enlarged end view of the ring lens employed in FIG. 11;

FIG. 13 is an enlarged cross-sectional side elevational view of a ring lens for an optical viewing device in accordance with an alternate embodiment of the present invention;

FIG. 14 is an enlarged cross-sectional side elevational view of a ring lens for an optical viewing device in accordance with still another alternate embodiment of the present invention;

FIG. 15 is an enlarged cross-sectional side elevational view of a ring lens for an optical viewing device in accordance with still yet another alternate embodiment of the present invention; and

FIG. 16 is a graphical representation of time domain multiplexing according to aspects of the disclosure.

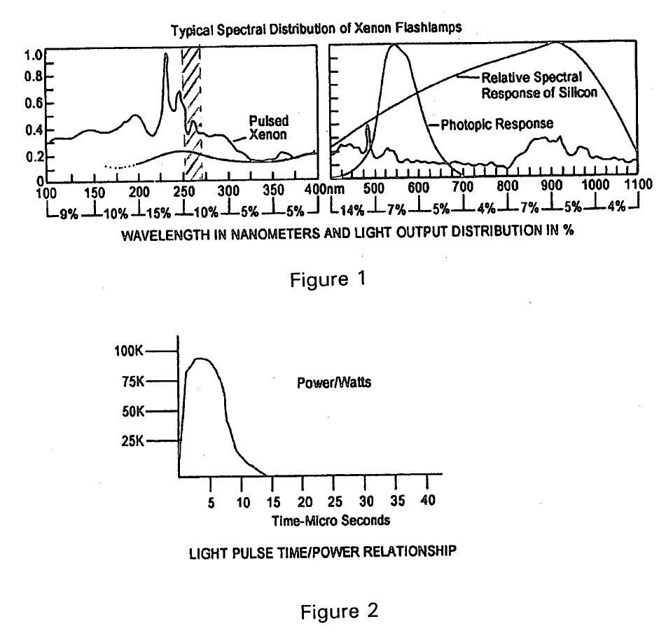

### DETAILED DESCRIPTION OF THE PREFERRED EMBODIMENTS

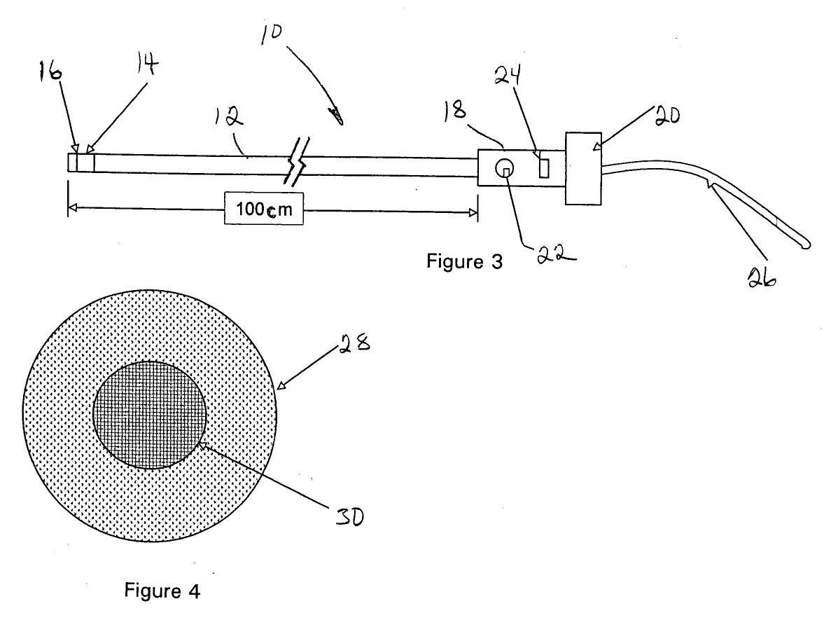

FIG. 3 is an exterior view of an endoscope for therapeutic light delivery according to aspects of the present invention. The endoscope 10 includes a probe 12 having an outside diameter of approximately 5 mm and a length of approximately 100 cm. The probe 12 terminates at a steerable distal end 14 and a light distribution optic 28 configured to radiate light from the distal end of the probe in a pre-determined pattern. The body 18 of the endoscope may house an electronic camera 20, a joystick or similar control 22 for steering the distal end of the probe and a control 24 for interrupting selected wavelengths of light delivered to the distal end of the probe. The illustrated endoscope probe is approximately 100 cm in length, flexible and steerable for use in the gastrointestinal tract as is known in the art. Other probe configurations and features may be necessary in other use environments. For example, an endoscope for use in nasal passages would have a diameter of about 3 mm and a shorter length of about 10 cm to 20 cm.

A service cable 26 includes optical fibers selected for transmission of therapeutic light and illumination light to the distal end of the endoscope probe. The service cable 26 also includes wires for powering the electronic components of the endoscope and retrieving image signals from the endoscope camera 20. The service cable 26 communicates with a service module (not shown) which may house a xenon flash lamp (or other) light source, power supplies, image processing electronics and may include a viewing screen for viewing images produced by the endoscope camera. A xenon flash lamp may alternatively be referred to as a pulsed xenon light source in this application. It will be understood that the xenon flash lamp is not a continuously operated arc lamp, but is a gaseous discharge lamp that produces short, intense bursts or pulses of light under electronic control. Each burst or pulse of light is followed by a dark period of no light emission. An aspect of the present invention relates to time domain multiplexing to employ the light delivery optical pathway of the endoscope for therapeutic as well as the typical imaging function.

FIG. 4 is an enlarged distal end view of a representative endoscope probe, showing the light distribution optic 28 surrounding the imaging fibers or rod 30 at the center of the probe. The light distribution optic 28 may be a Fresnel-type lens, employing internal reflection and/or refraction to produce a cylindrical light pattern as shown in FIG. 8. An aspect of the present invention relates to a light distribution optic configured to enhance light distribution from the endoscope probe in a radial direction to maximize delivery of therapeutic light to tissues and mucosa lining various body cavities and/or passages. This is accomplished by re-directing light from the optical fibers of the imaging bundle to a radial direction using internal reflection and refraction in the light distribution optic 28.

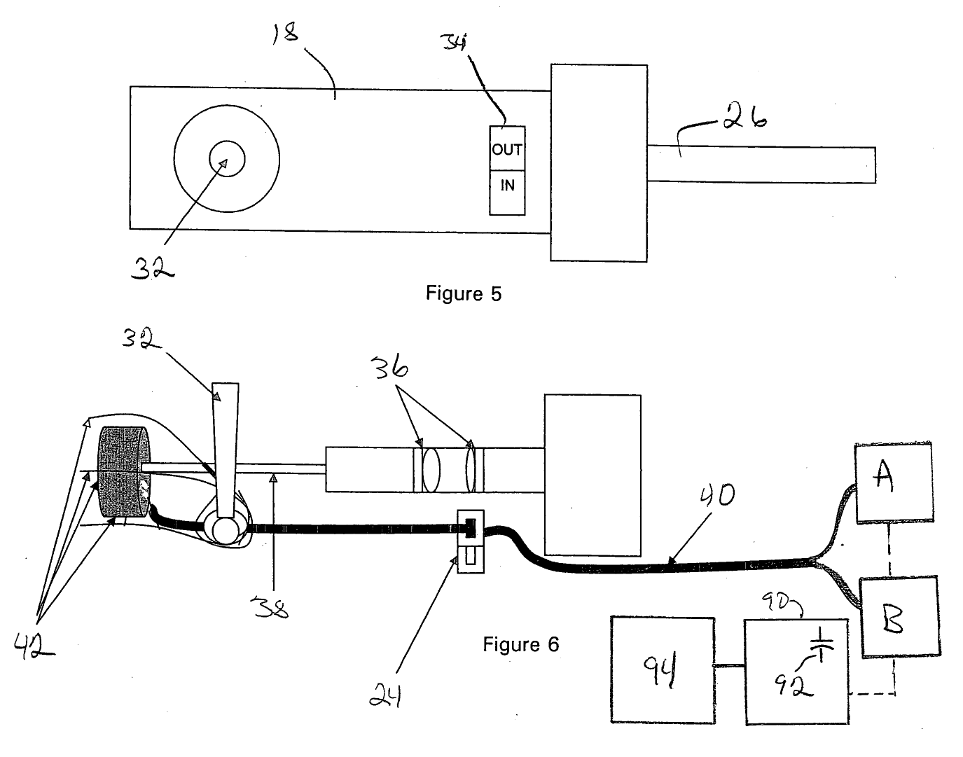

A further aspect of the present invention relates to a steerable distal end of the endoscope. FIG. 5 is an enlarged exterior view of an exemplary endoscope body illustrating a joystick-type control mechanism 32 for steering the probe distal end and a light filter 34 for control of light delivery to the probe distal end. The exemplary embodiment of the endoscope includes a steering control in the form of a joystick 32 and guide wires 42 that allow a user to steer the distal end of the endoscope in a known manner. A steerable distal end permits the endoscope to be guided along the gastrointestinal tract or other body passage or cavity. Other control mechanisms are known and may be compatible with the present invention.

FIG. 6 is an interior view of the endoscope body of FIG. 5, showing components of an exemplary endoscope 10. Imaging lenses 36 optically couple imaging fiber optics 38 to an electronic camera 20. A light control mechanism 24 may be arranged to interrupt the light delivery optical fiber bundle 40. In the case of therapeutic light in the ultraviolet wavelength (UV), the light control mechanism 24 may take the form of a filter arranged to prevent UV light from continuing along the light delivery optical fibers. The light delivery optical fibers are selected to be transmissive of the desired therapeutic wavelength. For example, delivery of therapeutic light in the ultraviolet wavelength, e.g., wavelengths between about 200 nm and about 350 nm, requires UV transmissive optical fibers. Optical fibers for UV transmission are produced by CeramOptec Industries, Inc. under the trademark Optran®. The fibers are silica, transmissive of 95% of the input in wavelengths from 160 to 1,200 nm. Other fibers and materials transmissive of UV are quartz, fused silica, some polymer fibers, UV glass, synthetic silica glass, and synthetic quartz fibers. Transmission of other light wavelengths may require selection of alternative optical fibers.

It is desirable to have control over delivery of the wavelengths of light delivered into the body. For example, UV can be damaging to sensitive tissues and its application should be selective. An aspect of the present invention relates to equipping the endoscope with apparatus for interrupting delivery of certain wavelengths of light, such as UV, while permitting other wavelengths of light, such as the visible spectrum, to pass.

In an alternative control arrangement shown in FIG. 6, an exemplary endoscope includes a control circuit 90 operatively connected to one or more light sources A, B. If the light source A, for example is a xenon flash lamp, the control circuit 90 will include a discharge capacitor 92 that is charged to a main discharge voltage by the control circuit. The capacitance in Farads of the discharge capacitor 92 and the value of the main discharge voltage are important factors in determining the power of the pulse of light produced by the xenon flash lamp. The power of the pulse of light produced by the xenon flash lamp is adjustable by the control circuit by varying the main discharge voltage.

An aspect of the present invention relates to a control interface 94, which allows a user to interact with the control circuit 90. Through the control interface 94, the user provides inputs to the control circuit 90 to adjust the power, frequency and wavelength of the light pulses produced by the light sources A, B. The quantity or dose of therapeutic light delivered to a target area can be precisely controlled by adjusting the energy content (power) of each light pulse, the number of light pulses generated per unit of time (frequency) and/or the spectrum (wavelengths) of light contained in each light pulse. A suitable control circuit 90 and control interface 94 facilitate this control. Light sources A, B may be used separately or in combination, depending upon the quantity, or dose of therapeutic light that is needed.

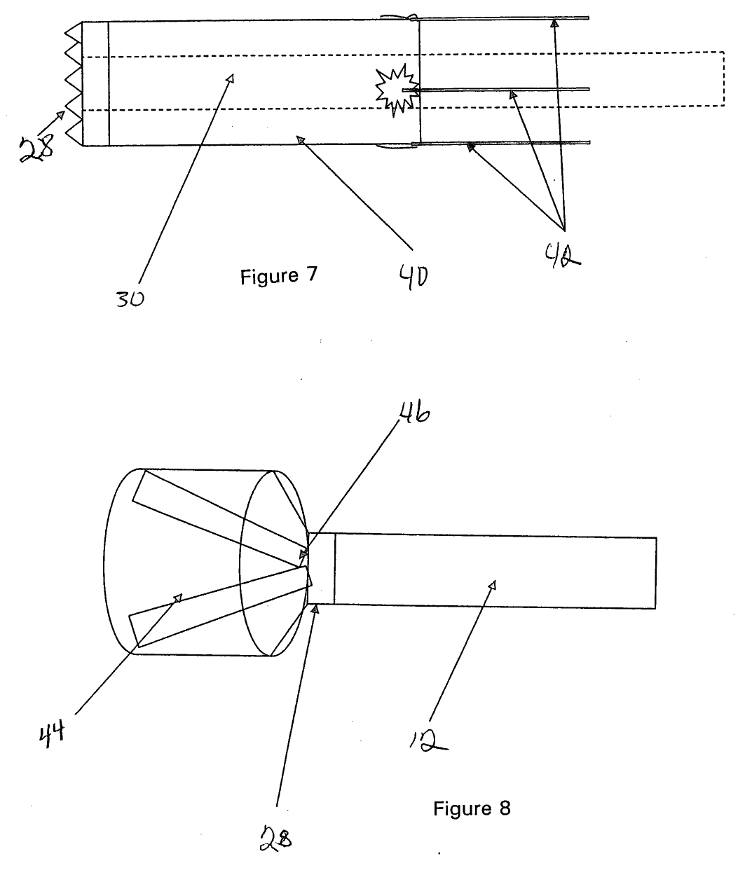

FIG. 7 is an enlarged sectional view through the distal end of the endoscope probe 12. The light distribution optic 28 forms a ring around the imaging channel 30. The imaging channel 30 may also be referred to as the image retrieval optical pathway. The light distribution optic 28 may be in the form of a lens positioned to spread light radially around the distal end of the endoscope. The imaging channel may be provided by an imaging rod or an optical fiber bundle optically coupled to an objective lens. FIG. 8 illustrates the distal end of the endoscope probe 12 and light distribution optic 28 with a representative cylindrical illumination pattern 44 and viewing field 46.

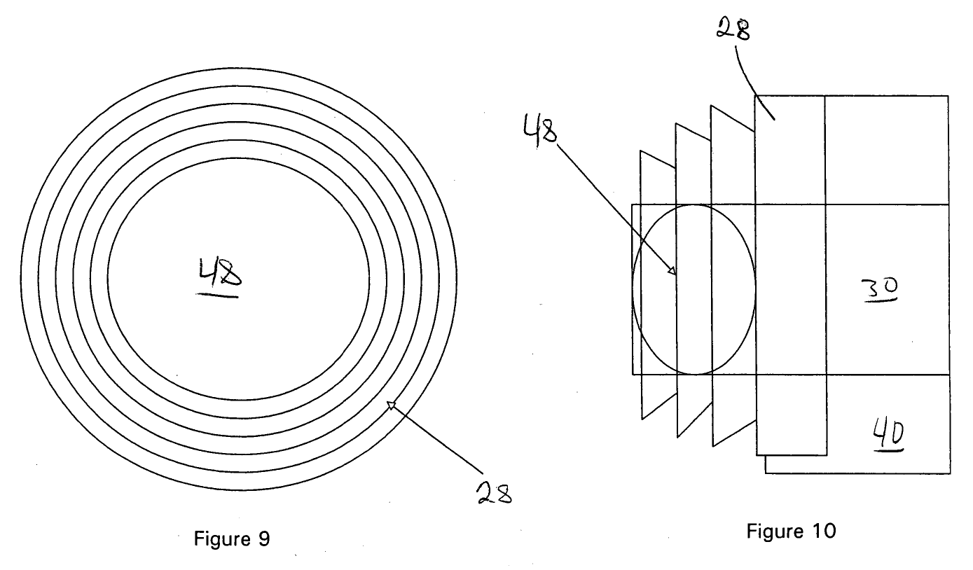

FIGS. 9 and 10 are enlarged end and sectional views of the probe distal end, showing an objective lens 48 for gathering image light and a Fresnel-type light distribution optic for distributing therapeutic light. The Fresnel-type illuminator is arranged to receive light from the light delivery optical fibers 40 and distribute the light in a cylindrical illumination pattern 44 as shown in FIG. 8. The objective lens 48 is configured to receive light from this cylindrical field and couple this image to the imaging fibers 40 at the center of the probe 12.

Also referring to FIGS. 2 and 3, the objective lens section 14 comprises six lenses including lenses 38-43.

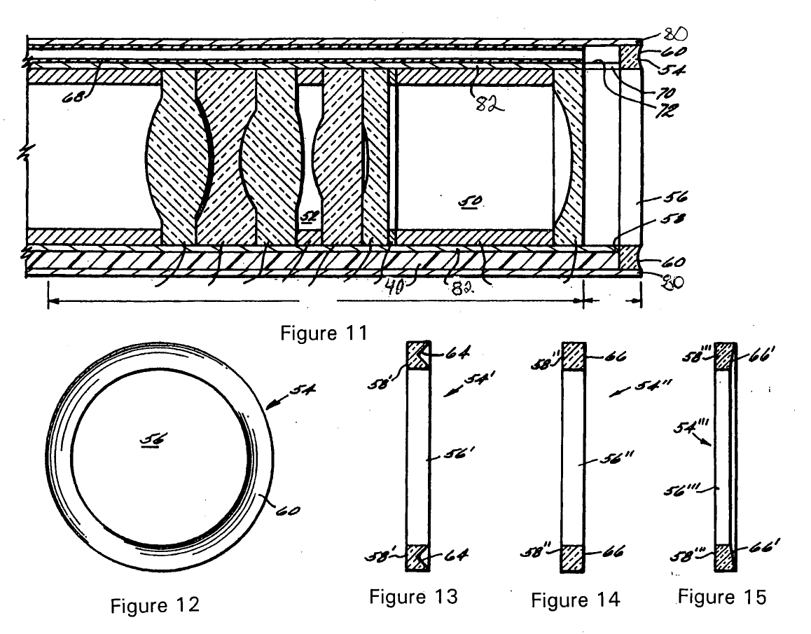

One example of a light distribution assembly is illustrated in FIGS. 11-15. The light distribution assembly comprises a ring shaped lens (i.e., an annular lens) 54 having a central opening 56 which aligns with lens 48 of the objective lens section. Unlike lens 48, lens 54 may be mounted within the outer tube 80 with inner tube 82 abutting an inner flat surface 58 of lens 54. Alternatively, lens 54 could be secured (e.g., by a suitable epoxy) to the fibers 40 and the inner and outer tubes 82, 80.

The present invention includes a means for illuminating the remote end (the end to the right of the objective lens section) so that light from this illuminating means may be reflected from the object to be viewed. According to aspects of the present invention, the light distribution optic is also employed to distribute therapeutic light to the area surrounding the distal end of the endoscope. In a preferred embodiment, the light distribution assembly includes a plurality of optical fibers 40 that are arranged in one or more layers along the inner circumference of outer tube 80. Optical fibers 40 are collected in a bundle in a body housing and attached to a commercially available and known fiber optic connector. A pulsed xenon light source is positioned at the terminal end of connector to provide light to the fiber optic bundle. In this way, light is delivered to the distal end 14 of the endoscope probe 12.

The optical fibers 40 may be comprised of a suitable polymeric material such as acrylic or polycarbonate materials, synthetic quartz, fused silica or other material transmissive of the desired light wavelengths. One problem with optical fibers is that the field of illumination may be relatively small because of their small numerical aperture and may therefore not be as large as desired for therapeutic purposes.

This problem has been overcome by lens 54, which has a front surface comprising a negative curvature 60. The fibers 40 align with the negative curvature 60 and are optically coupled with and physically attached to surface 58 of lens 56 by any well known means (e.g., by a suitable epoxy). Lens 54 is preferably a plastic lens comprised of a suitable optical grade plastic (e.g., a polymeric material). However, it is within the scope of the present invention that lens 54 be comprised of a optical grade glass. The negative curvature 60 increases the field of illumination to obtain a field of illumination that is appropriate for therapeutic purposes.

Referring to FIG. 13, in another embodiment, the ring shaped lens employs the lens design shown generally at 54′. Lens 54′ has a front surface comprising a double angled or wedge shape 64, a flat rear surface 58′ and a central opening 56′. Similar to the negative curvature 60 (FIG. 11) the wedge shape 64 of lens 54′ increases the field of illumination.

Referring to FIG. 14, in still another embodiment, the ring shaped lens employs the lens design shown generally at 54″. Lens 54″ has a flat front surface 66, a flat rear surface 58″ and a central opening 56″. However, with lens 54″ the polymeric optical fibers 24 are twisted as is described in U.S. Ser. Nos. 838,602 and 944,212. Such twisting significantly increase the field of illumination to obtain a field of illumination.

Referring to FIG. 15, is still yet another embodiment, the ring shaped lens employs the lens design shown generally at 54′″. Lens 54′″ has an angled or prism-like shape 66′, a flat rear surface 58′″ and a central opening 56′″. Similar to the negative curvature 60 (FIG. 11) the angled shape 66′ of lens 54′″ increases the field of illumination to obtain a field of illumination.

Since light delivery is emphasized, the cross-sectional area of light delivery material in the probe relative to the cross-sectional area of the image optics in the center of the probe may be greater than is typical in an imaging endoscope. The combination of enhanced light delivery and light spreading optics are selected to provide therapeutically significant light emission at the distal end of the endoscope probe.

An aspect of the present invention relates to control over delivery of the therapeutic light spectrum to the treatment site inside the body. As previously discussed, particular ranges of spectrum can be interrupted using a movable filter 24. An alternative arrangement is illustrated in FIG. 6. Pulsed xenon light sources A and B are each optically coupled to a subset of the light delivery optical fibers 40. As an example, pulsed xenon light source A provides the therapeutic spectrum and pulsed xenon light source B is arranged to produce light in the visible spectrum for imaging purposes. Light source B is triggered at a frequency above approximately 30 Hz to provide a steady image. Light source A is triggered under computer control to deliver the desired quantity of therapeutic spectrum. Therapeutic light from light source A can be can be delivered between pulses of light source B. Computer control over the power and frequency of light pulses from light source A allow precise control over the quantity of therapeutic spectrum delivered.

The intensity of the pulsed xenon light sources allow each light source to use less than the total available light delivery fibers and still provide sufficient illumination at the distal end of the probe. The relative proportions of the light delivery fibers assigned to each function (therapy or imaging) can be calculated according to the particular needs of the intended use.

One particularly useful range of wavelengths is in the UV range of between about 200 nm to about 300 nm and more particularly between about 250 nm to about 270 nm. This range of UV wavelengths is very effective at killing and/or disabling microorganisms such as fungi, bacteria and protozoa. The xenon flash lamp light source produces a strong emission in these wavelengths. Advantageously, the UV content of the light pulse produced by a xenon flash lamp typically increases along with the applied discharge voltage.

To ensure delivery of an effective dose of UV to kill or disable a pathogen of interest, the xenon flash lamp is activated at a rate of at least 30 Hz and preferably at a rate of approximately 60 Hz. However, the effective dose will depend upon many factors, including the pathogen in question, the properties of the target area, the power of each light pulse in the UV wavelengths, etc. Experimentation has indicated that endoscopic delivery of UV is effective at killing or disabling a wide variety of pathogens including bacteria and fungi including Pseudomonas aeruginosa, Acnetobacter, Staphylococcus aureus, Klebsiella Escherichia coli, Bacillus subtilis, Helicobacter pylori, and Aspergillus fumigates. Experimentation has also indicated that endoscopically delivered UV from a pulsed xenon light source can penetrate liquids to a depth of at least 3 mm-15 mm and about 3 mm through tissues such as human skin. It is believed that the peak intensity of the energy from the pulsed xenon light source enhances the penetration of the therapeutic light.

Light sources A, B shown in FIG. 6 may alternatively be lasers. Lasers also produce very intense light, although typically in very narrow band widths. The lasers would be selected to produce specific therapeutic wavelengths and operatively connected to the light delivery optical pathway of the endoscope. The control circuit 90 and interface 94 allow a user to control activation of the laser or lasers to provide measured doses of therapeutic light to a target area.

Another potential use for the inventive endoscope is photodynamic therapy. Photodynamic Therapy involves the application of a photosensitizing drug such as 5-aminolevulinic acid (5-ALA) followed by activation with light to produce a photodynamic effect. The most commonly used wavelengths are 640 nm (red light) and 400-450 nm (blue light). After topical application, the thermophotosensitizing drug preferentially accumulates in tumor and dysplastic cells, and is converted into the photosensitizer protoporphyrin IX (PpIX.) When activated by light, PpIX generates cytotoxic reactive oxygen species that selectively destroy cells, and may cause malignant and nonmalignant hyperproliferative tissue to be destroyed or to decrease in size.

Recent studies of laparoscopic fluorescence suggest that in vivo fluorescence may improve the early detection of intraperitoneal ovarian carcinoma micrometastases. In vivo fluorescence has also been used to detect occult gastrointestinal tumors, as well as peritoneal colon carcinoma metastases that were, previously, undetected. Fluorescence-based laparoscopy has also provided improved diagnostic accuracy in the staging of hepatocellular carcinoma, particularly in patients potentially suitable for partial liver resection or transplantation. It has also been used for in vivo detection of metatastic ovarian cancer in a rat model. In this study, tumor-free peritoneum did not show fluorescence, and was significantly distinguishable from cancer nodules. Embodiments of the disclosed endoscope could be equipped for endoscopic fluorescence.

Considerable research documents UV capability to destroy bacteria. Recent studies of bacillus anthracis (Anthrax) spores demonstrated significant inactivation when exposed to appropriate UV wavelengths.

Current research has demonstrated the value of utilizing IR illumination during thoracoscopic excision of mediastinal bronchogenic cysts to more easily identify the esophagus and to clarify the dissection plane between the cysts and the esophagus.

Pulsed xenon provides a simple and efficient light source for the activation of photodynamic diagnostic dyes than is currently available. As noted, this light source is extremely rich in UV and can generate sufficient narrow-band wavelengths to activate the Photo-dynamic and Photo-fluorescent dyes, while providing visual imaging capability simultaneously through a single device. In some cases, use of the multispectral therapeutic endoscope may eliminate the need to utilize a laser with its potential to damage tissue adjacent to the targeted site.

Until recently, UV wavelengths have only been routinely available during open surgery when a “Woods Light” has been used. Aspects of the present invention relating to multispectral endoscopy permit this capability to be used during closed procedures as well.

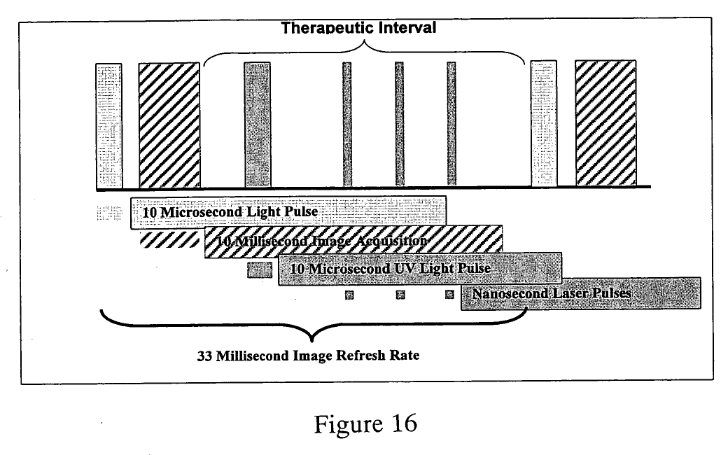

Dark space is a term used to describe the period of time between light pulses generated by the pulsed xenon light source. (See FIG. 16). Approximately 10 μS are required to illuminate the body cavity with light. Another 3 mS are necessary to read out the image from the CCD camera and update a visual monitor. The next light pulse occurs approximately 30 mS later. The time between is dark if no therapeutic intervention laser and/or light is being applied. If therapeutic intervention is being applied, the system synchronizes the interventional sources during this dark space.

Not all intervention and diagnostic imaging occurs in the visible spectrum. In fact, most therapeutic intervention occurs using infrared (IR) as well as UV radiation. The present system utilizes a light source that is active from approximately 200-1100 nm. As indicated previously in the example relating to Photodynamic therapy, wavelengths of 450 and 640 are used for dye activation. Other UV and IR wavelengths excite fluorescence in the visible. A broad spectrum light source is necessary to cover the full range of applications. In many cases, a filter is used to pass only a given band of wavelengths. The high energy output of the flashtube provides high peak energies throughout the UV, VIS and IR spectrums. The flash lamp can generate over 100,000 watts of peak power with each pulse. Since the pulse is only a few μS in duration, the average power consumed by the light source is less than 60 watts.

Alternatively, the therapeutic wavelengths may be supplied by lasers as shown above in Figure A. Lasers produce narrow band, focused light that can be ideal for destruction of targeted tissues or growths. Laser light can be delivered through a dedicated portion of the light delivery optical pathway.

Sensors and many activated materials are capable of reacting to these very short light pulses. In some cases however, a greater number pulses may be necessary to activate a given dye. The pulsed xenon light source provides controlled high-energy pulses of light for both imaging and therapeutic intervention across a broad optical spectrum.

The amount of energy that can be applied into the body cavity is precisely controlled by several independent means. The amount of energy released by the flash lamp is a function voltage applied to a capacitor discharged though the electrodes of the device. The energy follows the equation of:
E=½ CV where C=Capacitance in Micro Farads and V=the applied voltage

The xenon flash lamp power supply can be controlled from 400 to 1000 volts. This control may take the form of a D/A output from a computer into the power supply which then convert converts the low voltage signal to a high voltage signal with a DC to DC power converter.

A second means of control is increasing or decreasing the pulse rate, thereby providing an increasing or decreasing number of pulses. The maximum number of pulses is a function of the capacitor “C” as indicated in the above equation and the applied voltage.

A third means of controlling the pulses is to combine the output of multiple xenon flash lamps and/or lasers to increase the amount of energy applied to a given therapeutic function. The control circuit 90 can be equipped with outputs can be controlled to insert or remove an optical filter from either or both xenon flash lamp light sources, thereby providing a gross control.

All of the above control means are directed from a system computer control circuit 90 that establishes the operating conditions based on the surgical procedure and energy necessary for intervention.

While a preferred embodiment of the foregoing invention has been set forth for purposes of illustration, the foregoing description should not be deemed a limitation of the invention herein. Accordingly, various modifications, adaptations and alternatives may occur to one skilled in the art without departing from the spirit and the scope of the present invention.

What is claimed is:

1. A method for delivery of therapeutic light comprising:
providing an endoscope with a light delivery optical pathway transmissive of said therapeutic light, said endoscope also including an image retrieval optical pathway and imaging system for generating an image of a target area;
providing a light generator that selectively produces said therapeutic light and also generates visible light;
inserting said endoscope into a cavity of a living organism to identify and illuminate a target area, said inserting including employing the image to direct said insertion and identify said target area;
activating said light generator to produce said therapeutic light to achieve a therapeutic objective at said target area; and
removing said endoscope from said cavity.

2. The method of claim 1, wherein said step of providing a light generator comprises providing a light generator that selectively produces therapeutic UV light having a wavelength of between 200 nm and 300 nm.

3. The method of claim 1, wherein said step of providing a light generator comprises providing a xenon flash lamp that selectively produces therapeutic UV light having a wavelength of between 200 nm and 300 nm.

4. The method of claim 1, wherein said therapeutic objective is to kill microscopic pathogens with UV light.

5. The method of claim 1, comprising the step of:
multiplexing the generation of visible light for image production with the production of said therapeutic light; and
delivering said visible light and said therapeutic light to the target area through the light delivery optical pathway at different times.

6. The method of claim 1, wherein said cavity is the nasal cavity of a human.

7. An endoscope comprising:
a light delivery optical pathway constructed of materials selected to transmit light including UV light having a wavelength between 200 nm and 300 nm;
a light distribution optic arranged to receive light from said light delivery optical pathway and distribute said light in a pre-determined pattern, said light distribution optic constructed of materials selected to be transmissive of said UV light;
an image retrieval optical pathway;
a high intensity pulsed light source functionally arranged to deliver pulses of broad spectrum light to said light delivery optical pathway, said pulses including imaging pulses and therapeutic pulses;
a light control mechanism arranged to selectively prevent light having a wavelength less than about 400 nm from reaching said light distribution optic; and
an image generating system which employs light from said image pulses incident upon a target area and gathered by said image retrieval optical pathway to generate an image of said target area,
wherein said image pulses are generated according to a regular pattern and said therapeutic pulses are selectively generated between said image pulses.

8. The endoscope of claim 7, wherein said high intensity light source is a Xenon flash lamp.

9. The endoscope of claim 7, wherein said light control mechanism comprises a filter moveable to block light having a frequency below about 400 nm from entering said light delivery optical pathway.

10. The endoscope of claim 7, wherein said pre-determined pattern includes distributing light in a radial direction surrounding a distal end of said endoscope.

11. The endoscope of claim 7, wherein said light distribution optic comprises a Fresnel-type lens.

12. A. therapeutic light delivery endoscope comprising:
a broad spectrum light source that generates pulses of light having wavelengths between about 190 nm and about 1100 nm;
a control circuit operatively connected to said broad spectrum light source, said control circuit providing adjustable control over the frequency, power and wavelength of said light pulses;
a light delivery optical pathway constructed of materials selected to transmit light including UV light having a wavelength between 200 nm and 300 nm, said light delivery optical pathway arranged to receive and transmit light generated by said broad spectrum light source to a target area;
an image retrieval optical pathway arranged to receive light reflected from said target area;
an image generating system which employs light from said image retrieval optical pathway to generate an image of said target area; and
an interface allowing a user to adjust the frequency, power and wavelength of said pulses of light, thereby controlling the quantity of said UV light delivered to said target area.

13. The therapeutic light delivery endoscope of claim 12, wherein adjustment of the wavelength of said light pulses is adjusted by a filter responsive to said control circuit.

14. The therapeutic light delivery endoscope of claim 12, wherein said broad spectrum light source comprises a first light source which generates light having a wavelength above about 400 nm and a second light source generating light having a wavelength between about 200 nm and about 300 nm, the wavelength of said light pulses is adjusted by selectively energizing said first and second light sources.

15. The therapeutic light delivery endoscope of claim 12, wherein said broad spectrum light source is a xenon flash lamp.

16. The therapeutic light delivery endoscope of claim 15, wherein said control circuit includes a discharge capacitor having a capacitance and charges said capacitor with a main discharge voltage, the power of each said high-intensity pulse of broad spectrum light being responsive to the value of said capacitance and said main discharge voltage, said control circuit adjusting the power of each said pulse of broad spectrum light by variation of said main discharge voltage according to inputs received from said interface.

17. The therapeutic light delivery endoscope of claim 15, wherein said control circuit is responsive to inputs received from said interface to determine the number of light pulses containing light having a wavelength of below about 300 nm that are delivered to said light delivery optical pathway.

18. The therapeutic light delivery endoscope of claim 12, wherein said light pulses include image light pulses generated according to a timing pattern and said image generating system is arranged to detect light from said image retrieval optical pathway based on said timing pattern, said timing pattern including a delay between image light pulses, said light pulses further including therapeutic light pulses delivered during said delay, said therapeutic light pulses containing light having a wavelength of below about 300 nm.

19. The therapeutic light delivery endoscope of claim 12, wherein said light delivery optical pathway is constructed from UV transmissive materials selected from quartz, UV glass, and synthetic silica glass.

20. The therapeutic light delivery endoscope of claim 12, wherein said light delivery optical pathway includes a light distribution optic arranged to radiate said light in a pre-determined pattern, said light distribution optic disposed at a distal end of said endoscope.
